(19) United States
(12) Patent Application Publication
(10) Pub. No.: US 2012/0107278 A1
(43) Pub. Date: May 3, 2012

(54) ABBREVIATED HCV THERAPY FOR HCV INFECTED PATIENTS WITH IL28B C/C GENOTYPE

(75) Inventors: MIRIAM MICHELLE BERREY, Durham, NC (US); Peter Schaefer Price, JR., Princeton, NJ (US); William T. Symonds, Cary, NC (US)

(73) Assignee: Pharmasset, Inc., Princeton, NJ (US)

(21) Appl. No.: 13/285,444

(22) Filed: Oct. 31, 2011

Related U.S. Application Data
(60) Provisional application No. 61/408,304, filed on Oct. 29, 2010.

(51) Int. Cl. A61K 38/21; C12Q 1/70; A61P 31/14

(52) U.S. Cl. 424/85.7; 435/5

(57) ABSTRACT

Disclosed herein is a method for administering an abbreviated hepatitis C virus (HCV) treatment regimen for an HCV-infected, rs12979860 single nucleotide polymorphism (SNP) (C/C) patient, which comprises: administering to said patient an effective amount of each of a direct-acting antiviral, pegylated interferon alfa-2a, and ribavirin; wherein the abbreviated HCV treatment regimen is less than 48 weeks. Also disclosed herein is a method of detecting an rs12979860 single nucleotide polymorphism (SNP) in a hepatitis C virus (HCV) infected patient comprising: detecting the rs12979860 SNP of chromosome 19 in a biological sample from a patient, wherein an rs12979860 SNP C/C patient is administered an abbreviated treatment regimen with a direct-acting antiviral agent in combination with peginterferon alfa-2a and ribavirin; and wherein the abbreviated HCV treatment regimen is less than 48 weeks.

### PRIORITY

The present application claims priority to U.S. Provisional Patent Application 61/408,304, filed on Oct. 29, 2010.

### FIELD OF THE INVENTION

Disclosed herein are methods for treating HCV infected patients with an IL28B C/C genotype.

### BACKGROUND

Hepatitis C virus (HCV) infection is a major health problem that leads to chronic liver disease, such as cirrhosis and hepatocellular carcinoma, in a substantial number of infected individuals, estimated to be 2-15% of the world's population. There are an estimated 4.5 million infected people in the United States alone, according to the U.S. Center for Disease Control. According to the World Health Organization, there are more than 200 million infected individuals worldwide, with at least 3 to 4 million people being infected each year. Once infected, about 20% of people clear the virus, but the rest can harbor HCV the rest of their lives. Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their offspring. Current treatments for HCV infection, which are restricted to immunotherapy with recombinant interferon-α alone or in combination with the nucleoside analog ribavirin, are of limited clinical benefit. Moreover, there is no established vaccine for HCV. Consequently, there is an urgent need for improved therapeutic agents that effectively combat chronic HCV infection.

The HCV virion is an enveloped positive-strand RNA virus with a single oligoribonucleotide genomic sequence of about 9600 bases which encodes a polyprotein of about 3,010 amino acids. The protein products of the HCV gene consist of the structural proteins C, E1, and E2, and the non-structural proteins NS2, NS3, NS4A and NS4B, and NS5A and NS5B. The nonstructural (NS) proteins are believed to provide the catalytic machinery for viral replication. The NS3 protease releases NS5B, the RNA-dependent RNA polymerase from the polyprotein chain. HCV NS5B polymerase is required for the synthesis of a double-stranded RNA from a single-stranded viral RNA that serves as a template in the replication cycle of HCV. Therefore, NS5B polymerase is considered to be an essential component in the HCV replication complex (K. Ishi, et al, Heptology, 1999, 29: 1227-1235; V. Lohmann, et al., Virology, 1998, 249: 108-118). Inhibition of HCV NS5B polymerase prevents formation of the double-stranded HCV RNA and therefore constitutes an attractive approach to the development of HCV-specific antiviral therapies.

The current standard of care (SOC) for the treatment of chronic HCV infection is a combination therapy with pegylated interferon alfa-2a (Pegasys®) or pegylated interferon alfa-2b)(PegIntron®, both in combination with ribavirin (Copegus®). The primary goal of treatment for chronic hepatitis C is a sustained virologic response (SVR), which has been previously defined as a patient completing at least 24 weeks post treatment in a state free of viral RNA as measured in accordance with the assay methodology described below. SVR is also described in detail by Dr. Steven L. Flamm in the Journal of the American Medical Association, Vol. 289, No. 18, pp. 2413 to 2417. Host factors including age, body weight, race, and advanced fibrosis influence outcome of treatment (Dienstag and McHutchison Gastroenterology 2006; 130: 231-264 and Missiha et al. Gastroenterology 2008; 134: 1699-1714), but are poor predictors of response. In contrast, viral factors like the genotype and the on-treatment pattern of viral response can be used to determine the likelihood of treatment success and guide treatment duration individually and proved to be very useful in clinical practice. Ge et al. Nature (2009) 461: 399-401.

In spite of encouraging response in some patients when subjected to these various treatments, the response among patients infected with Hepatitis C virus is approximately 50%, particularly amongst the patients infected with genotype 1 HCV. There is also a need to provide therapy which reduces the time that patients show evidence of complete viral suppression (negative HCV status) following the initiation of treatment.

Accordingly, there remains a need for a treatment methodology which quickly brings the patients' viral load to an undetectable level, which in turn enhances in patients the achievement of a sustained viral response (SVR). There further remains a need for a treatment methodology that reduces the likelihood of developing treatment resistant strains of HCV. What is needed also is method of identifying patients having a high likelihood of successful outcome using an HCV treatment methodology that reduces the likelihood of a patient developing treatment resistant strains of HCV and increasing the likelihood of achieving an SVR.

### SUMMARY

Disclosed herein is a method for administering an abbreviated hepatitis C virus (HCV) treatment regimen for an HCV-infected, rs12979860 single nucleotide polymorphism (SNP) (C/C) patient, which comprises: administering to said patient an effective amount of each of a direct-acting antiviral, pegylated interferon alfa-2a, and ribavirin; wherein the abbreviated HCV treatment regimen is less than 48 weeks.

Also disclosed herein is a method of detecting an rs12979860 single nucleotide polymorphism (SNP) in a hepatitis C virus (HCV) infected patient comprising: detecting the rs12979860 SNP of chromosome 19 in a biological sample from a patient, wherein an rs12979860 SNP C/C patient is administered an abbreviated treatment regimen with a direct-acting antiviral agent in combination with peginterferon alfa-2a and ribavirin; and wherein the abbreviated HCV treatment regimen is less than 48 weeks.

### BRIEF DESCRIPTION OF THE DRAWINGS

FIG. 1: Week 6 HCV RNA (IU/mL) for patients having a mean PSI-6206 trough concentration (in ng/mL) for varying dosages 100 mg PSI-7977 QD (♦), 200 mg PSI-7977 QD (▴), and 400 mg PSI-7977 QD ().

FIG. 2A: PSI-7977 100 mg+SOC individual patient variation in HCV RNA during the course of the study.

FIG. 2B: PSI-7977 200 mg+SOC individual patient variation in HCV RNA during the course of the study.

FIG. 2C: PSI-7977 400 mg+SOC individual patient variation in HCV RNA during the course of the study.

FIG. 3: Patient 1 variation in HCV RNA during the course of the study.

FIG. 4: Patient 2 variation in HCV RNA during the course of the study.

### DETAILED DESCRIPTION OF THE INVENTION

### Definitions/Abbreviations

The term “effective amount” as used herein means an amount required to reduce symptoms of the HCV infection in a subject.

A sustained virologic response (SVR) for a patient is defined as a patient who completes the abbreviated HCV treatment regimen of a direct-acting antiviral in combination with pegylated interferon alfa-2a, and ribavirin and who is free of HCV RNA (LOD<15 IU/mL) for at least 24 weeks post treatment as measured in accordance with the assay methodology described below.

RVR is the abbreviation for rapid virologic response. The occurrence of RVR is predictive of ultimate SVR with a full treatment course of 48 weeks in HCV GT-1 patients (Poordad, F., et al., Clin. Infect. Dis. 2008 46: 78-84.)

QD means that the dose is administered once a day.

QW means that the dose is administered once a week.

The highest activities of alanine aminotransferase (ALT) are found in hepatocytes and striated (skeletal and cardiac) muscle cells. Increased serum ALT activity can accompany hepatocellular injury or necrosis of striated muscle. With cell injury or death, ALT escapes from the cytosol. In addition, release of ALT from the cytosol can occur secondary to cellular necrosis or as a result of cellular injury with membrane damage. Determination of ALT activity is a relatively sensitive indicator of hepatic damage. Mechanisms of increased activity of ALT in serum include enzyme release from damaged cells or induction of enzyme activity, such as increased enzyme synthesis from drug administration. (Zeuzem, S., et al., Aliment Pharmacol Ther. 2006 Oct. 15; 24(8) 1133-1149).

The interleukin 28B (IL28B) gene encodes a cytokine distantly related to type I interferons and the IL-10 family. The IL28B gene, interleukin 28A (IL28A), and interleukin 29 (IL29) are three closely related cytokine genes that form a cytokine gene cluster on a chromosomal region mapped to 19q13. Expression of the cytokines encoded by the three genes can be induced by viral infection. All three cytokines have been shown to interact with a heterodimeric class II cytokine receptor that consists of interleukin 10 receptor, beta (IL10RB), and interleukin 28 receptor, alpha (IL28RA). (National Center for Biotechnology Information, Entrez Gene Entry for IL28B, Gene ID: 282617, updated on 23 Oct. 2010).

The body mass index (BMI) is a measurement based on a person's weight and height and is used to estimate a healthy body weight based on a person's height, assuming an average body composition. The units of BMI are kg/m².

HOMA-IR is the homeostatic model assessment for insulin resistance, and is used to quantify insulin resistance and beta-cell function.

IFN is the abbreviation for interferon.

LOD is the abbreviation for limit of detection.

GT is the abbreviation for genotype.

IU is the abbreviation for international unit, which is a measure of the biological activity of a substance.

CI is the abbreviation for confidence interval.

WNL is the abbreviation for within normal limits.

The nucleotides present in DNA are adenine (A), thymine (T), cytosine (C), and guanine (G).

A single nucleotide polymorphism (SNP) is a DNA sequence variation that occurs when a single nucleotide in the genome differs between members of a species or paired chromosomes in an individual. As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases (Stephens et al. Science 293, 489-493, 20 Jul. 2001). See U.S. Pat. No. 7,820,380, which is incorporated by reference.

The term “detecting” is used in the broadest sense to include both qualitative and quantitative measurements of a specific molecule, for example, measurements of a specific molecule such as a chromosome, a DNA sequence, individual nucleic acids, etc.

The term “biological sample” refers to a body sample from any animal, but preferably is from a mammal, more preferably from a human. Such samples include biological fluids such as serum, plasma, vitreous fluid, lymph fluid, synovial fluid, follicular fluid, seminal fluid, amniotic fluid, whole blood, biopsy material, urine, cerebro-spinal fluid, saliva, sputum, tears, perspiration, and mucus. The term also refers to tissue extracts, such as homogenized tissue and cellular extracts, and cellular samples (e.g., oral epithelial cells).

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder.

### Embodiments

A first embodiment is directed to a method for administering an abbreviated hepatitis C virus (HCV) treatment regimen for an HCV-infected, rs12979860 single nucleotide polymorphism (SNP) (C/C) patient, which comprises: administering to said patient an effective amount of each of a direct-acting antiviral, pegylated interferon alfa-2a, and ribavirin; wherein the abbreviated HCV treatment regimen is less than 48 weeks.

In a first aspect of the first embodiment, the administering occurs from about 2 weeks to about 12 weeks.

In a second aspect of the first embodiment, the administering occurs from about 2 weeks to about 6 weeks.

In a third aspect of the first embodiment, the administering occurs from about 2 weeks to about 4 weeks.

In a fourth aspect of the first embodiment, the administering occurs for about 4 weeks.

In a fifth aspect of the first embodiment, the administering occurs for about 2 weeks.

In a sixth aspect of the first embodiment, wherein the patient is infected with HCV genotype (GT) 1, HCV GT 2, HCV GT 4, or HCV GT 5.

In a seventh aspect of the first embodiment, the patient is infected with HCV GT 1.

In an eighth aspect of the first embodiment, the direct-acting antiviral is PSI-7977.
Additional aspects of the first embodiment include the rs12979860 SNP C/C patient being administered with:
   100 mg once per day of PSI-7977, 180 μg once per week of peginterferon alfa-2a, and 1000 mg once per day of ribavirin;
   100 mg once per day of PSI-7977, 180 μg once per week of peginterferon alfa-2a, and 1200 mg once per day of ribavirin;
   200 mg once per day of PSI-7977, 180 μg once per week of peginterferon alfa-2a, and 1000 mg once per day of ribavirin;
   200 mg once per day of PSI-7977, 180 μg once per week of peginterferon alfa-2a, and 1200 mg once per day of ribavirin;
   400 mg once per day of PSI-7977, 180 μg once per week of peginterferon alfa-2a, and 1000 mg once per day of ribavirin; 400 mg once per day of PSI-7977, 180 μg once per week of peginterferon alfa-2a, and 1000 mg once per day of ribavirin for about two weeks to about six weeks;
   400 mg once per day of PSI-7977, 180 μg once per week of peginterferon alfa-2a, and 1000 mg once per day of ribavirin for about two weeks to about six weeks wherein upon the end of the administration period the rs12979860 SNP (C/C) patient has an HCV RNA level less than about 15 IU/mL;
   400 mg once per day of PSI-7977, 180 μg once per week of peginterferon alfa-2a, and 1000 mg once per day of ribavirin wherein the administration lasts for about 2 weeks to about four weeks;
   400 mg once per day of PSI-7977, 180 μg once per week of peginterferon alfa-2a, and 1000 mg once per day of ribavirin wherein the administration lasts for about 2 weeks to about four weeks and wherein upon the end of the administration period the rs12979860 SNP (C/C) patient has an HCV RNA level less than about 15 IU/mL;
   400 mg once per day of PSI-7977, 90 μg to 180 μg once per week of peginterferon alfa-2a, and 1000 mg or 1200 mg once per day of ribavirin;
   400 mg once per day of PSI-7977, 90 μg to 180 μg once per week of peginterferon alfa-2a, and 1000 mg or 1200 mg once per day of ribavirin wherein the administration lasts for about two weeks to about six weeks;
   400 mg once per day of PSI-7977, 90 μg to 180 μg once per week of peginterferon alfa-2a, and 1000 mg or 1200 mg once per day of ribavirin wherein the administration lasts for about two weeks to about six weeks wherein 180 μg of peginterferon alfa-2a is administered in the first two weeks;
   400 mg once per day of PSI-7977, 90 μg to 180 μg once per week of peginterferon alfa-2a, and 1000 mg or 1200 mg once per day of ribavirin wherein the administration lasts for about two weeks to about six weeks wherein 180 μg of peginterferon alfa-2a is administered in the first two weeks and wherein 90 μg of peginterferon alfa-2a is administered during weeks 3 through 6;
   400 mg once per day of PSI-7977, 90 μg to 180 μg once per week of peginterferon alfa-2a, and 1000 mg or 1200 mg once per day of ribavirin wherein the administration lasts for about two weeks to about four weeks;
   400 mg once per day of PSI-7977, 90 μg to 180 μg once per week of peginterferon alfa-2a, and 1000 mg or 1200 mg once per day of ribavirin wherein the administration lasts for about two weeks to about four weeks. and wherein 180 μg of peginterferon alfa-2a is administered in the first two weeks;
   400 mg once per day of PSI-7977, 90 μg to 180 μg once per week of peginterferon alfa-2a, and 1000 mg or 1200 mg once per day of ribavirin;
   400 mg once per day of PSI-7977, 90 μg to 180 μg once per week of peginterferon alfa-2a, and 1000 mg or 1200 mg once per day of ribavirin wherein the administration lasts for about two weeks to about six weeks, and wherein 180 μg of peginterferon alfa-2a is administered in the first two weeks. wherein 90 μg of peginterferon alfa-2a is administered during weeks 3 through 4; and
   400 mg once per day of PSI-7977, 180 μg once per week of peginterferon alfa-2a, and 1000 mg or 1200 mg once per day of ribavirin for two weeks followed by 400 mg once per day of PSI-7977, 90 μg once per week of peginterferon alfa-2a, and 1000 mg or 1200 mg once per day of ribavirin for two weeks.

A second embodiment is directed to a method of detecting an rs12979860 single nucleotide polymorphism (SNP) in a hepatitis C virus (HCV) infected patient comprising: detecting the rs12979860 SNP of chromosome 19 in a biological sample from a patient, wherein an rs12979860 SNP C/C patient is administered an abbreviated treatment regimen with a direct-acting antiviral agent in combination with peginterferon alfa-2a and ribavirin; and wherein the abbreviated HCV treatment regimen is less than 48 weeks.

In a first aspect of the second embodiment, the rs12979860 SNP C/C patient is administered from about 2 weeks to about 12 weeks.

In a second aspect of the second embodiment, the rs12979860 SNP C/C patient is administered from about 2 weeks to about 6 weeks.

In a third aspect of the second embodiment, the rs12979860 SNP C/C patient is administered from about 2 weeks to about 4 weeks.

In a fourth aspect of the second embodiment, the rs12979860 SNP C/C patient is administered for about 4 weeks.

In a fifth aspect of the second embodiment, the rs12979860 SNP C/C patient is administered for about 2 weeks.

In a sixth aspect of the second embodiment, the rs12979860 SNP C/C patient is infected with HCV genotype (GT) 1, HCV GT 2, HCV GT 4, or HCV GT 5.

In a seventh aspect of the second embodiment, the rs12979860 SNP C/C patient is infected with HCV GT 1.

In an eighth aspect of the second embodiment, the rs12979860 SNP C/C patient is administered PSI-7977.
Additional aspects of the second embodiment include the rs12979860 SNP C/C patient being administered with:
   100 mg once per day of PSI-7977, 180 μg once per week of peginterferon alfa-2a, and 1000 mg once per day of ribavirin;
   100 mg once per day of PSI-7977, 180 μg once per week of peginterferon alfa-2a, and 1200 mg once per day of ribavirin;
   200 mg once per day of PSI-7977, 180 μg once per week of peginterferon alfa-2a, and 1000 mg once per day of ribavirin;
   200 mg once per day of PSI-7977, 180 μg once per week of peginterferon alfa-2a, and 1200 mg once per day of ribavirin;
   400 mg once per day of PSI-7977, 180 μg once per week of peginterferon alfa-2a, and 1000 mg once per day of ribavirin;
   400 mg once per day of PSI-7977, 180 μg once per week of peginterferon alfa-2a, and 1000 mg once per day of ribavirin for about two weeks to about six weeks;
   400 mg once per day of PSI-7977, 180 μg once per week of peginterferon alfa-2a, and 1000 mg once per day of ribavirin for about two weeks to about six weeks wherein upon the end of the administration period the rs12979860 SNP (C/C) patient has an HCV RNA level less than about 15 IU/mL;
   400 mg once per day of PSI-7977, 180 μg once per week of peginterferon alfa-2a, and 1000 mg once per day of ribavirin wherein the administration lasts for about 2 weeks to about four weeks;
   400 mg once per day of PSI-7977, 180 μg once per week of peginterferon alfa-2a, and 1000 mg once per day of ribavirin wherein the administration lasts for about 2 weeks to about four weeks and wherein upon the end of the administration period the rs12979860 SNP (C/C) patient has an HCV RNA level less than about 15 IU/mL;
   400 mg once per day of PSI-7977, 90 μg to 180 μg once per week of peginterferon alfa-2a, and 1000 mg or 1200 mg once per day of ribavirin;
   400 mg once per day of PSI-7977, 90 μg to 180 μg once per week of peginterferon alfa-2a, and 1000 mg or 1200 mg once per day of ribavirin wherein the administration lasts for about two weeks to about six weeks;
   400 mg once per day of PSI-7977, 90 μg to 180 μg once per week of peginterferon alfa-2a, and 1000 mg or 1200 mg once per day of ribavirin wherein the administration lasts for about two weeks to about six weeks wherein 180 μg of peginterferon alfa-2a is administered in the first two weeks;
   400 mg once per day of PSI-7977, 90 μg to 180 μg once per week of peginterferon alfa-2a, and 1000 mg or 1200 mg once per day of ribavirin wherein the administration lasts for about two weeks to about six weeks wherein 180 μg of peginterferon alfa-2a is administered in the first two weeks and wherein 90 μg of peginterferon alfa-2a is administered during weeks 3 through 6;
   400 mg once per day of PSI-7977, 90 μg to 180 μg once per week of peginterferon alfa-2a, and 1000 mg or 1200 mg once per day of ribavirin wherein the administration lasts for about two weeks to about four weeks;
   400 mg once per day of PSI-7977, 90 μg to 180 μg once per week of peginterferon alfa-2a, and 1000 mg or 1200 mg once per day of ribavirin wherein the administration lasts for about two weeks to about four weeks. and wherein 180 μg of peginterferon alfa-2a is administered in the first two weeks;
   400 mg once per day of PSI-7977, 90 μg to 180 μg once per week of peginterferon alfa-2a, and 1000 mg or 1200 mg once per day of ribavirin;
   400 mg once per day of PSI-7977, 90 μg to 180 μg once per week of peginterferon alfa-2a, and 1000 mg or 1200 mg once per day of ribavirin wherein the administration lasts for about two weeks to about six weeks, and wherein 180 μg of peginterferon alfa-2a is administered in the first two weeks. wherein 90 μg of peginterferon alfa-2a is administered during weeks 3 through 4; and
   400 mg once per day of PSI-7977, 180 μg once per week of peginterferon alfa-2a, and 1000 mg or 1200 mg once per day of ribavirin for two weeks followed by 400 mg once per day of PSI-7977, 90 μg once per week of peginterferon alfa-2a, and 1000 mg or 1200 mg once per day of ribavirin for two weeks.

### Direct Acting Antivirals

Examples of a “direct-acting antiviral” (“DAA”) include, but are not limited to: (see EP 1881001, US 2003/0187018, US 2005/0267018, US 2003/0119752, US 2003/0187018, US 2005/0090432, US 2009/0291902, US 2005/0267018, US 2005/0267018, US 2011/0237621, US 2009/0281141, US 2009/0105302, US 2009/0062311, US 2009/0281140, US 2007/0054842, US 2008/0108617, and US 2008/0108617); HCV NS5B Inhibitors (see US 2004/0229840, US 2005/0154056, US 2005/0098125, US 2006/0194749, US 2006/0241064, US 2006/0293306, US 2006/0040890, US 2006/0040927, US 2006/0166964, US 2007/0275947, U.S. Pat. No. 6,784,166, US 2007/0275930, US 2002/0147160, US 2002/0147160, US 2003/0176433, US 2004/0024190, US 2005/0043390, US 2005/0026160, US 2004/0171570, US 2005/0130923, US 2008/0146788, US 2007/0123484, US 2007/0024277, US 2007/0004669, US 2004/0142989, US 2004/0142993, US 2006/0004063, US 2006/0234962, US 2007/0231318, US 2007/0142380, WO 2004/096210, US 2007/0135363, WO 2005/103045, US 2008/0021047, US 2007/0265222, US 2006/0046983, US 2008/0280842, WO 2006065590, US 2006/0287300, WO 2007039142, WO 2007039145, US 2007/0232645, US 2007/0232627, WO 2007088148, WO 2007092000, and US 2010/0234316); HCV NS4 Inhibitors (see US 2005/0228013 and US 2007/0265262); HCV NS5A Inhibitors (see US 2006/0276511, US 2007/0155716, US 2008/0182863, US 2009/0156595, and US 2008/0182863); Toll-like receptor agonists (see US 2007/0197478); and other inhibitors (see US 2003/0207922, US 2006/0094706, US 2006/0122154, US 2005/0069522, US 2005/0096364, US 2005/0069522, US 2005/0096364, and US 2005/0215614); PSI-6130 (U.S. Pat. No. 7,429,572); RG7128 (U.S. Pat. No. 7,754,699); Compound A (disclosed in US 2010/0081628, see also compound 19a (PSI-938) and 19b disclosed in the same application, which are individual diastereomers of compound A); PSI-7977 (U.S. Pat. No. 7,964,580, claim 8) and PSI-7976 (disclosed in US 2010/0016251 and US 2010/0298257 (Ser. No. 12/783,680) (PSI-7977 (Sp-4) and PSI-7976 (Rp-4)); PSI-353661 (disclosed in US 2010/0279973, see compound II); telaprevir (also known as VX-950, which is disclosed in US 2010/0015090); boceprevir (disclosed in US 2006/0276405); BMS-790052 (disclosed in US 2008/0050336, see also US 2009/0041716); ITMN-191 (disclosed in US 2009/0269305 at Example 62-1); ANA-598 (shown below and identified as compound 31 in F. Ruebasam et al. Biorg. Med. Chem. Lett. (2008) 18: 3616-3621; and TMC435 (formerly known as TMC435350). Each of these patent and non-patent documents are incorporated by reference.

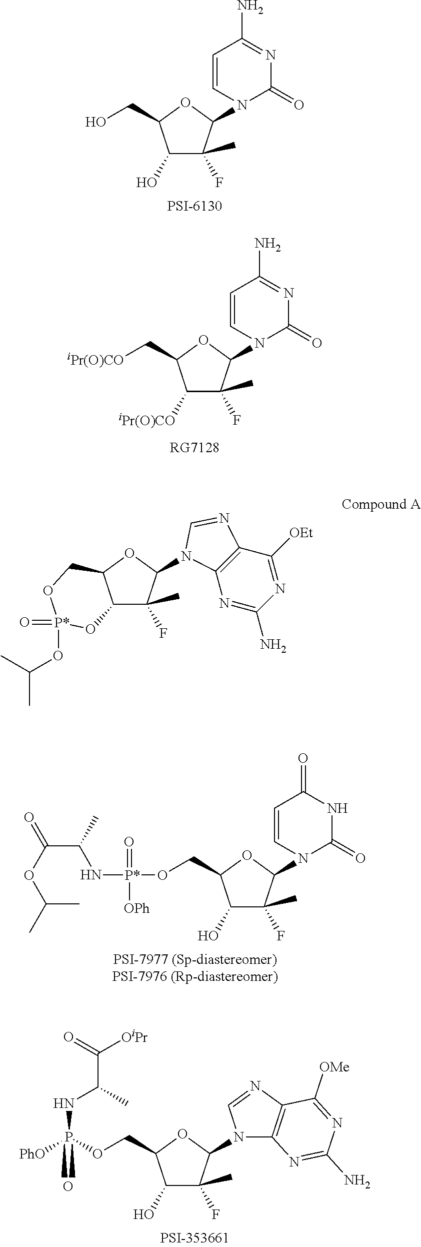

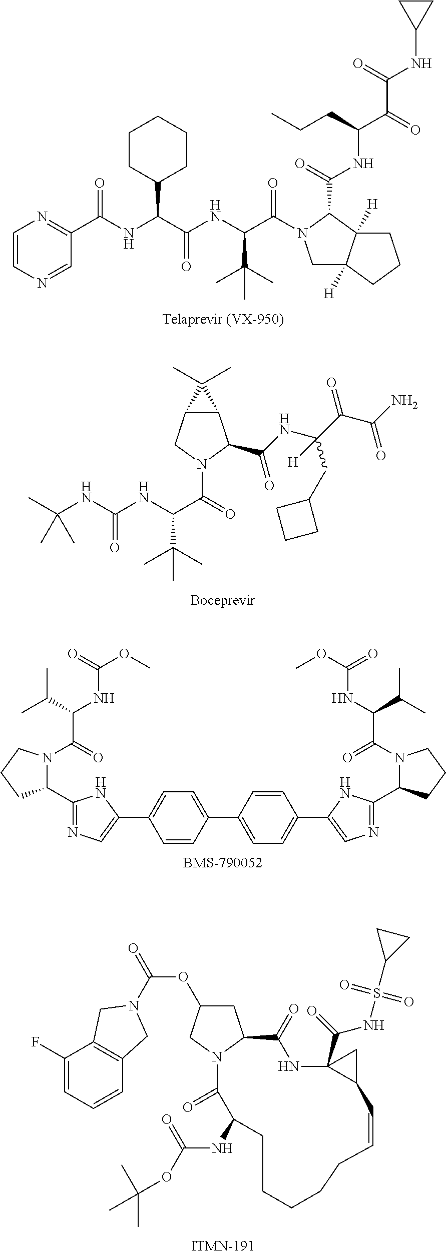

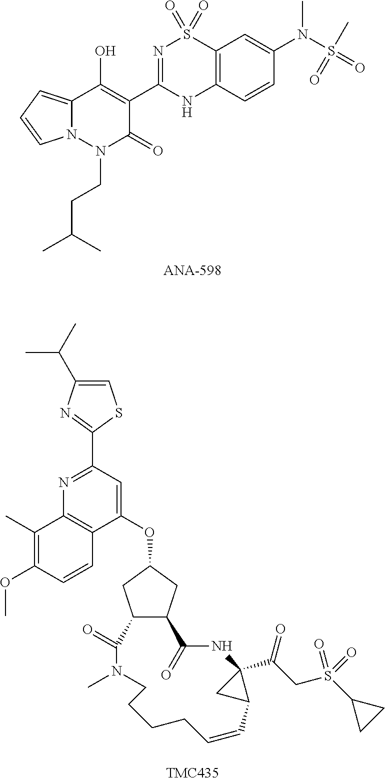

The antiviral agents can be formulated in a manner known to one of ordinary skill. The respective patent documents provide guidance for the respective formulations. The preferred dosage forms of the antiviral agents are those that are approved by the FDA. However, not to be limited, contemplated dosage forms of the antiviral agents are contemplated as follows: RG7128 (500 mg, 1000 mg, or 1500 mg); Compound A (50 mg to 1000 mg and values inbetween); PSI-7977 (100 mg, 200 mg, or 400 mg); A dosage form for VX-950 is disclosed in McHutchison et al. N. Engl. J. Med. (2009) 360(18): 1827-1838; see also WO 2009/038663.); Boceprevir (WO 2009/038663).

Additional direct-acting antivirals and contemplated dosages are identified in the following table.

| | | | Clinical | |
|---|---|---|---|---|
| Drug Name | Drug Category | Company | Phase | Dosage |
| RG7128 | Polymerase | Roche in | Phase I | 500 mg |
| | Inhibitor | collaboration with | | BID, 100 mg |
| | | Pharmasset | | BID |
| RG7227 | Protease | Roche in | Phase I | 100 mg TID, |
| | Inhibitor | collaboration with | | 200 mg TID |
| | | Pharmasset | | |
| Telaprevir | Protease | Vertex | Phase II | N/A |
| (VX-950) | Inhibitor | | | |
| VX-222 | Polymerase | Vertex | Phase II | N/A |
| | Inhibitor | | | |
| BMS 790052 | NS5a Inhibitor | Bristol-Myers Squibb | Phase II | 60 mg once a |
| | | | | day or 600 |
| | | | | mg twice a |
| | | | | day |
| BMS 65032 | Protease | Bristol-Myers Squibb | Phase II | 60 mg once a |
| | Inhibitor | | | day or 600 |
| | | | | mg twice a |
| | | | | day |
| BMS-824393 | NS5A Inhibitor | Bristol-Myers Squibb | Phase I | N/A |
| INX-189 | HCV Polymerase | Inhibitex | Phase I | from 3 mg to |
| | Inhibitor | | | 100 mg, once |
| | | | | a day |
| PSI-938 | Polymerase | Pharmasset | Phase I | TBD |
| | Inhibitor | | | |
| PPI-461 | NS5A Inhibitor | Presidio | Phase I | four single |
| | | Pharmaceuticals | | doses |
| | | | | followed by |
| | | | | a 5-day, |
| | | | | once-a-day |
| | | | | dose |
| IDX375 | Polymerase | Idenix | Phase I | 25 mg once |
| | Inhibitor | | | daily (QD), |
| | | | | 50 mg QD, |
| | | | | 100 mg QD, |
| | | | | 200 mg QD, |
| | | | | or 200 mg |
| | | | | twice a day |
| ABT-072 | Polymerase | Abbott | Phase I | N/A |
| | Inhibitor | | | |
| Clemizole | NS4B Inhibitor | Eiger | Phase I | N/A |
| | | BioPharmaceuticals | | |
| MK-3281 | Polymerase | Merck | Phase I | N/A |
| | Inhibitor | | | |
| PSI-7851 | Polymerase | Pharmasset | Phase I | 50 mg, |
| | Inhibitor | | | 100 mg, |
| | | | | 200 mg, or |
| | | | | 400 mg |
| ABT-450 | Protease | Abbott/Enanta | Phase I | N/A |
| HCV | Inhibitor | | | |
| VX-813 | Protease | Vertex | Phase I | N/A |
| | Inhibitor | | | |
| PHX1766 | Protease | Phenomix | Phase I | 400 mg BID |
| | Inhibitor | | | or 800 mg |
| | | | | BID |
| ABT-333 | Polymerase | Abbott | Phase I | N/A |
| | Inhibitor | | | |
| VX-916 | HCV Polymerase | Vertex | Phase I | N/A |
| | Inhibitor | | | |
| RG7128 | Polymerase | Pharmasset/Genentech | Phase I | 500 or |
| | Inhibitor | | | 100 mg BID |
| VX-500 | HCV Protease | Vertex | Phase I | N/A |
| | Inhibitor | | | |
| Filibuvir | HCV Polymerase | Pfizer | Phase II | 200, 300, or |
| (PF- | Inhibitor | | | 0500 mg |
| 00868554) | | | | BID (twice a |
| | | | | day) |
| ACH-1625 | Protease | Achillion | Phase II | 200 or 600 |
| | Inhibitor | | | mg |
| GS-9256 | Protease | Gilead | Phase II | N/A |
| | Inhibitor | | | |
| BI 201335 | Protease | Boehringer Ingelheim | Phase II | 240 mg |
| | Inhibitor | Pharma | | (once-a-day) |
| | | | | or 240 mg |
| | | | | (twice-a-day) |
| VX-222 | Polymerase | Vertex | Phase II | 250, 500, or |
| | Inhibitor | | | 750 mg |
| | | | | twice-a-day; |
| | | | | 1500 mg |
| | | | | once-a-day |
| RG7227 | Protease | InterMune/Genentech | Phase II | N/A |
| (Danoprevir) | Inhibitor | | | |
| ANA598 | Polymerase | Anadys | Phase II | First day 800 |
| | Inhibitor | Pharmaceuticals | | mg BID, |
| | | | | followed by |
| | | | | 200 or 400 |
| | | | | mg twice |
| | | | | daily |
| Vaniprevir | HCV Protease | Merck | Phase II | 300 or 600 |
| (MK-7009) | Inhibitor | | | mg twice a |
| | | | | day; 300 or |
| | | | | 600 mg |
| | | | | once-a-day |
| A-832 | NS5A Inhibitor | ArrowTherapeutics | Phase II | N/A |
| GS 9190 | Polymerase | Gilead | Phase II | N/A |
| | Inhibitor | | | |
| VX-759 | Polymerase | Vertex | Phase II | 400 mg TID, |
| | Inhibitor | | | 800 mg BID, |
| | | | | or 800 mg |
| | | | | TID |
| SCH900518 | Protease | Schering/Merck | Phase II | N/A |
| (Narlaprevir) | Inhibitor | | | |
| BI 207127 | Polymerase | Boehringer Ingelheim | Phase II | N/A |
| | Inhibitor | Pharma | | |
| PSI-7977 | Polymerase | Pharmasset | Phase IIa | 100, 200, or |
| | Inhibitor | | | 400 mg |
| | | | | once-a-day |
| TMC435 | Protease | Medivir/Tibotec | Phase IIa | N/A |
| | Inhibitor | | | |
| BMS 791325 | Polymerase | Bristol-Myers Squibb | Phase IIa | N/A |
| | Inhibitor | | | |
| BMS 650032 | Protease | Bristol-Myers Squibb | Phase IIa/b | N/A |
| | Inhibitor | | | |
| BMS 790052 | NS5a Inhibitor | Bristol-Myers Squibb | Phase IIb | N/A |
| Boceprevir | Protease | Schering | Phase III | 800 mg three |
| (SCH | Inhibitor | | | times a day |
| 503034) | | | | |
| Telaprevir | Protease | Vertex | Phase III | 750 mg |
| (VX 950) | Inhibitor | | | every 8 |
| | | | | hours; 1125 |
| | | | | mg dose |
| | | | | every 12 |
| | | | | hours; |
| BMS-824393 | Type Unknown | Bristol-Myers Squibb | Phase I | N/A |
| SCY-635 | Cyclophilin | SCYNEXIS | Phase I | up to 900 |
| | Inhibitor | | | mg/day |
| ANA773 | TLR Agonist | Anadys | Phase I | 800, 1200, |
| | | Pharmaceuticals | | 1600, or 200 |
| | | | | mg every |
| | | | | other day |
| CYT107 | Immunomodulator | Cytheris | Phase I | N/A |
| CF102 | A3AR Agonist | CAN-FITE | Phase I | N/A |
| IMO-2125 | TLR9 Agonist | Idera | Phase I | N/A |
| | | Pharmaceuticals | | |
| Bavituximab | Anti-Phospholipid | Peregrine | Phase I | N/A |
| (formerly | Therapy | | | |
| Tarvacin) | | | | |
| NOV-205 | Immunomodulator | Novelos Therapeutics | Phase I | N/A |
| SD-101 | TLR9 Agonist | Dynavax | Phase Ib | N/A |
| Miravirsen | microRNA | Santaris | Phase II | up to 12 |
| Formerly | | Pharma | | mg/kg |
| (SPC3649-LNA- | | | | |
| antimiR ™- | | | | |
| 122) | | | | |
| CTS-1027 | Anti- | Conatus | Phase II | N/A |
| | inflammatory | | | |
| Oglufanide | Immunomodulator | Implicit Bioscience | Phase II | N/A |
| disodium | | | | |
| Alinia | Thiazolides | Romark | Phase II | 500 mg |
| (nitazoxanide) | | | | twice daily |
| SCV-07 | Broad Spectrum | SciClone | Phase II | N/A |
| | Immune | | | |
| | Stimulator | | | |
| MitoQ | Inflammation/ | Antipodean | Phase II | N/A |
| (mitoquinone) | Fibrosis Inhibitor | Pharmaceuticals | | |
| Debio 025 | Cyclophilin | Debio | Phase II | 600 to 1000 |
| | Inhibitor | | | mg/day |
| PF-03491390 | Pancaspase | Pfizer | Phase II | 5 mg to 400 |
| (Formerly | Inhibitor | Pharmaceuticals | | mg daily |
| IDN-6556) | | | | (given 1 to 3 |
| | | | | times a day) |

According to the FDA-approved label dated Oct. 8, 2010, which is hereby incorporated by reference, the recommended dose of COPEGUS (ribavirin) tablets depends on body weight and the HCV genotype to be treated, as shown in the following table.

| HCV Genotype | PEGASYS Dose* | COPEGUS Dose | Duration |
|---|---|---|---|
| Genotypes 1, 4 | 180 μg | <75 kg = 1000 mg | 48 weeks |
| | | ≧75 kg = 1200 mg | 48 weeks |
| Genotypes 2, 3 | 180 μg | 800 mg | 24 weeks |

Genotypes 2 and 3 showed no increased response to treatment beyond 24 weeks.
*See PEGASYS Package Insert for further details on PEGASYS dosing and administration. The FDA-approved labels for PEGASYS dated Apr. 20, 2009 and May 24, 2010 are incorporated by reference.

The COPEGUS label further discloses that the recommended duration of treatment for patients previously untreated with ribavirin and interferon is 24 to 48 weeks. The daily dose of COPEGUS is 800 mg to 1200 mg administered orally in two divided doses. The dose should be individualized to the patient depending on baseline disease characteristics (e.g., genotype), response to therapy, and tolerability of the regimen.

### Examples

US 2010/0226885, which is incorporated by reference, discloses a method for measuring whether a patient has achieved an HCV negative status, e.g., at pp. 7-8 and throughout the disclosure.

We have followed with interest reports of direct acting antiviral (DAA) compounds in development for chronic hepatitis C (McHutchison et al. N Eng J Med 2009; 360:1827-1838 and Kwo et al., Lancet 2010; 376(9742):705-716.) and of genetic factors which predict interferon responsiveness. (Ge et al. Nature (2009) 461: 399-401.) We report two cases of Sustained Viral Response (SVR24) in patients with genotype 1 chronic hepatitis C infection (GT-1 HCV) who received compound 1 and pegylated interferon alfa-2a/ribavirin (standard of care, SOC) for 28 days. PSI-7977 is a novel uridine nucleotide analog with potent antiviral effects and no evidence of viral resistance in a 3-day monotherapy study (Rodriguez-Torres et al. American Society for the Study of Liver Diseases 2009: Latebreaker #17.), currently in Phase 2b in HCV GT 1-3. In a 28-day study to evaluate PSI-7977/SOC, 63 treatment-naïve patients with HCV GT-1 were stratified by IL28B status (rs12979860) (Ge et al. Nature (2009) 461: 399-401.) to receive PSI-7977 100 mg, 200 mg, 400 mg QD or placebo with SOC. At completion of 28 days subjects were offered 44 weeks additional SOC.

121 treatment-naïve HCV-infected patients were screened, and 63 patients enrolled across 7 sites in the U.S. No patients were excluded based on IL28B status. Patients were excluded for HCV RNA<100,000 IU/mL, non-1 HCV genotype, or cirrhosis. Patients were stratified into cohorts only by IL28B status: C/C vs. any T allele (C/T or T/T). The four cohorts included PSI-7977 100 mg, 200 mg, 400 mg QD or placebo with SOC for 28 days. Stratification based on only IL28B status did not result in treatment cohorts balanced for baseline HCV RNA or gender.

Of the 96 HCV genotype 1 patients, IL28B C/C status was confirmed in 23% (22 of 96) screened patients, and in 27% of enrolled subjects. Of the seven screening subjects excluded based on HCV RNA<15 IU/mL, 5 of the 7 were C/C. Over 80% of screened patients from Puerto Rico, Florida, or Texas were found to carry the T allele, versus 60% in San Francisco and Seattle. Although not stratified for other variables, the treatment groups were well-matched for age, sex, BMI, and baseline HCV RNA (˜6.5 log₁₀ IU/mL). Baseline HCV RNA for patients with C/C was 6.7 log₁₀ IU/mL, versus 6.4 log₁₀ IU/mL for patients with a T allele. PSI-7977 demonstrated potent antiviral activity with an RVR of 88-94% across all doses tested with no viral breakthrough. Five subjects self-identified as black; 4 of the 5 subjects were T/T and one was C/T. All five subjects achieved RVR on PSI-7977/SOC. No influence of IL28B genotype was evident in time to HCV RNA below the limit of detection. On placebo/SOC 3 of 14 (21%) achieved RVR: one each C/C, C/T, and T/T. Baseline HCV RNA was lower in the two patients with a T allele.

TABLE 1
Demographics With Only IL28B Stratification

| | 100 mg/ | 200 mg/ | 400 mg/ | Placebo/ |
|---|---|---|---|---|
| | SOC | SOC | SOC | SOC |
| | (n = 16) | (n = 18) | (n = 15) | (n = 14) |
| IL28B C/C | 4/16 | 5/18 | 4/15 | 4/14 |
| n/N (%) | (25) | (28) | (27) | (29) |
| Gender, F:M | 5:11 | 8:10 | 4:11 | 3:11 |
| BMI | 28 | 27 | 27 | 31 |
| mean (range) | (21-35) | (19-36) | (20-36) | (23-36) |
| Baseline HCV RNA | 6.64 | 6.28 | 6.49 | 6.48 |
| (log10 IU/mL) | | | | |
| Race | 1:15:0 | 2:16:0 | 2:12:1 | 0:14:0 |
| B/W/O | | | | |
| Ethnicity | 7:9 | 9:9 | 8:7 | 4:10 |
| Hispanic/non-H | | | | |
| HOMA-IR <3 | 9/16 | 13/18 | 7/15 | 7/14 |
| n/N (%) | (56) | (72) | (47) | (50) |

The proportion of the study population having the C/C genotype of the rs1299860 SNP was found to differ across HCV clinical trial sites, as shown in Table 2. Stratification may be important in early dose-ranging trials to avoid imbalance of interferon responsiveness.

TABLE 2
IL28B Distribution

| Clinical Trial Site | IL28B rs12979860 C/C Distribution n/N (%) | |
|---|---|---|
| Durham, North Carolina | 1/4 | (25%) |
| San Antonio, Texas | 5/31 | (16%) |
| San Francisco, California | 11/32 | (34%) |
| Santurce, Puerto Rico | 1/17 | (6%) |
| Seattle, Washington | 4/9 | (44%) |
| Orlando, Florida | 1/4 | (25%) |

The variation in HCV RNA in response to three different doses of PSI-7977 combined with SOC is shown in Table 3. The IL28B genotype had no effect on RVR or HCV RNA change from baseline in multivariate analyses. The only significant covariate effect was baseline HCV RNA.

TABLE 3
HCV RNA Results by PSI-7977 Dose

| | 100 mg/SOC | | 200 mg/SOC | | 400 mg/SOC | |
|---|---|---|---|---|---|---|
| | CC | CT/TT | CC | CT/TT | CC | CT/TT |
| | n = 4 | n = 12 | n = 5 | n = 13 | n = 4 | n = 11 |
| RVR, n/N | 4/4 | 10/12 | 4/5* | 13/13 | 4/4 | 10/11 |
| (%) | (100%) | (83%) | (80%) | (100%) | (100%) | (91%) |
| HCV RNA | −5.68 | −5.17 | −4.91 | −5.09 | −5.63 | −5.19 |
| Change from | (−4.79, −6.57) | (−4.75, −5.59) | (−3.32, −6.51) | (−4.62, −5.57) | (−4.87, −6.40) | (−4.78, −5.59) |
| Baseline at | | | | | | |
| Day 28 | | | | | | |
| (mean, log10 | | | | | | |
| [95% CI]) | | | | | | |
| Days to first | 18.8 | 19.5 | 14.0 | 15.8 | 18.7 | 14.0 |
| HCV RNA < | (2.92, 34.6) | (15.7, 23.3) | (1.14, 26.9) | (11.5, 20.0) | (8.63, 28.7) | (9.28, 18.7) |
| LOD | | | | | | |
| (mean, [95% | | | | | | |
| CI]) | | | | | | |

*One subject who was lost to follow-up prior to Day 28 is considered a failure

Genomic DNA was extracted using commercially available kits, and the rs1299860 SNP was determined at The Institute for Genome Science and Policy at Duke University. The genotype is also determined using the Illumina Human610-quad BeadChip, as outlined in Ge et al. Nature (2009) 461: 399-401, and references cited therein. HCV RNA was detected using the TaqMan® HCV Test (TaqMan® HCV; Roche Molecular Systems Inc., Branchburg, N.J.), which is also known as the 5′ nuclease assay (U.S. Pat. Nos. 5,210,015 and 5,538,848, see also U.S. Published Application No. 2006/0110724). The disclosure of U.S. Pat. No. 7,820,380, which is incorporated by reference, provides a detailed discussion on a variety of methods that employ SNP genotyping. For example, it is disclosed that Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al., Genet. Anal. Tech. App 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and 51 protection or chemical cleavage methods.

U.S. Pat. No. 7,820,380 also discloses that SNP genotyping can include the steps of, for example, collecting a biological sample from a human subject (e.g., sample of tissues, cells, fluids, secretions, etc.), isolating nucleic acids (e.g., genomic DNA, MRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

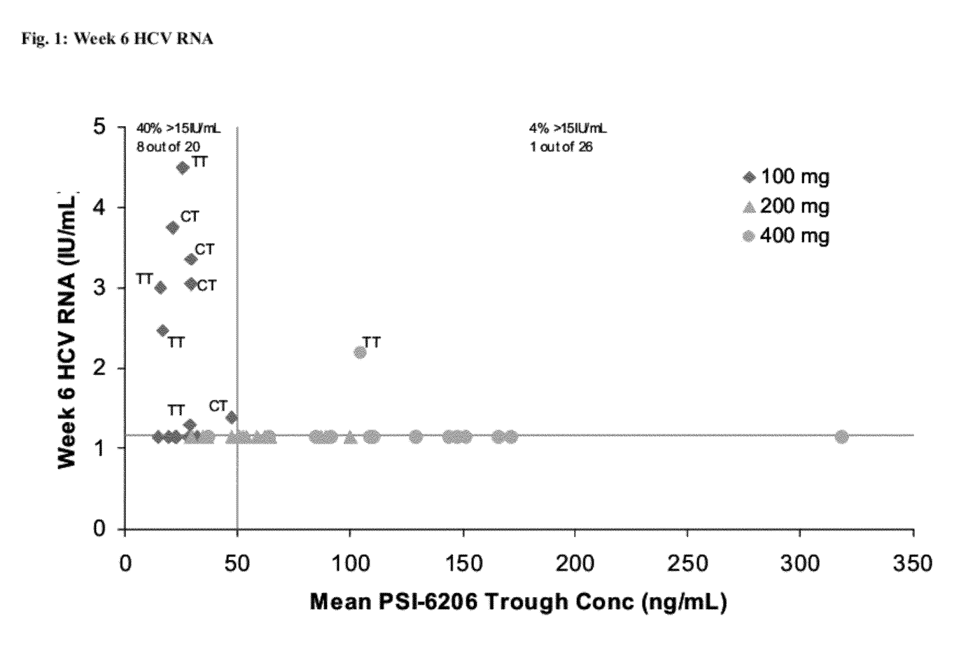

In week six of the study, eight out of twenty patients with the C/T or T/T genotype of the rs1299860 SNP had HCV RNA above the LOD (15 IU/mL). In contrast, all patients with the C/C genotype of the rs1299860 SNP had HCV RNA below the LOD (15 IU/mL). Higher systemic drug concentrations appeared to overcome “unfavorable” IL28B genotype (T/T) predictive of IFN-responsiveness (FIG. 1). PSI-6206 is a nucleoside metabolic by-product of PSI-7977.

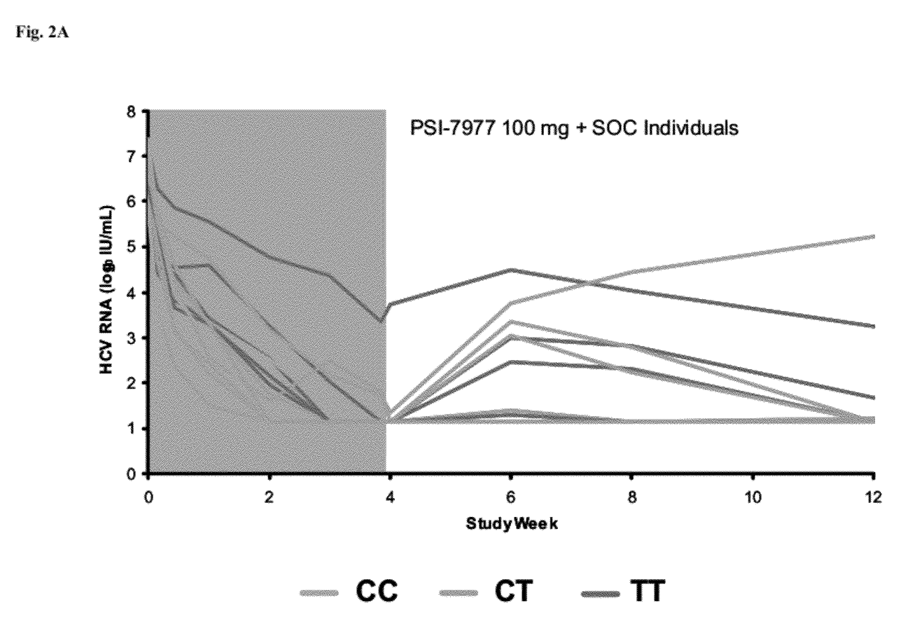

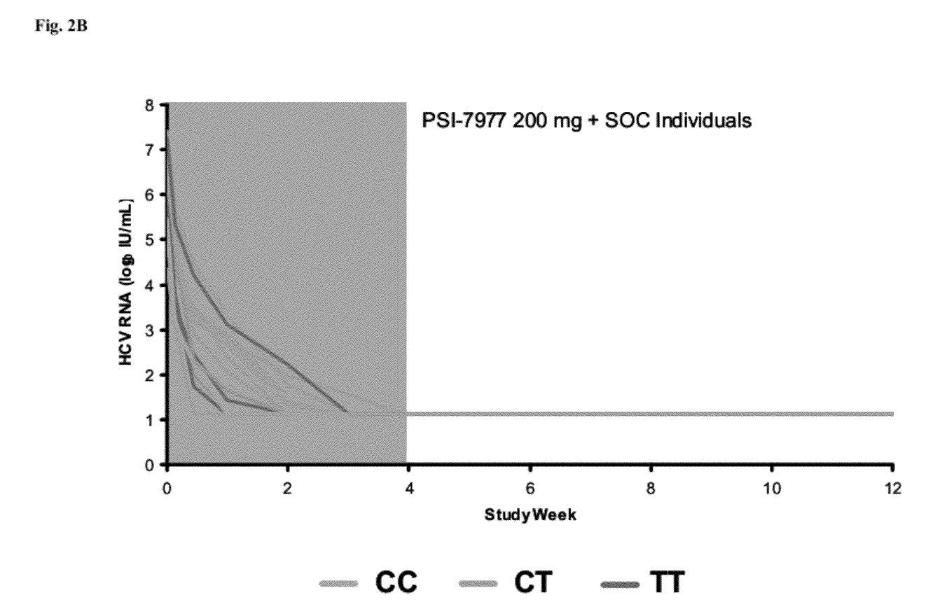

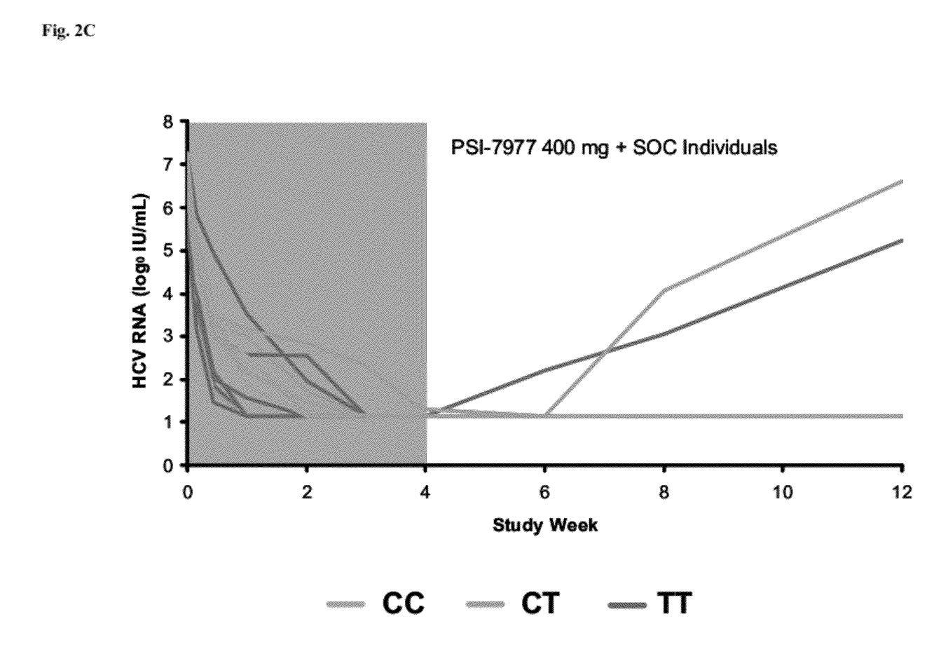

IL28B C/C subjects on PSI-7977 at all doses achieved RVR and maintained HCV RNA<LOD during SOC follow-up with no rebound (as shown in FIGS. 2A, 2B, and 2C).

Two of the 15 patients who received PSI-7977 400 mg/SOC declined to continue SOC after day 28 due to IFN-related intolerability.

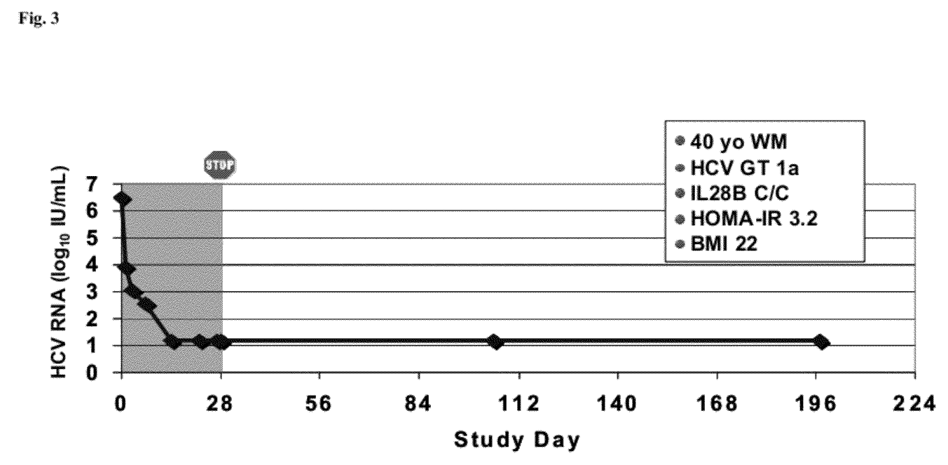

Patient 1, a 40 year old Caucasian male with GT-1a HCV, had high-level viremia (6.48 log₁₀ IU/mL), Stage 1 fibrosis, BMI 22 kg/m², HOMA-IR 3.2, and C/C IL28B polymorphism. Patient 1 was randomized to PSI-7977 400 mg QD for 28 days and also elected to discontinue PEG-IFN and RBV at day 28 due to interferon intolerance issues. He experienced a rapid antiviral response with HCV RNA below limit of detection (LOD, 15 IU/mL) at day 14 (as shown in FIG. 3). ALT activity was elevated (118 IU, Grade 1) at baseline and normalized by day 7. No dose reductions in SOC occurred prior to discontinuation of all therapy on day 28. HCV RNA remained <LOD through 24 weeks following 4 weeks of PSI-7977/SOC (SVR24).

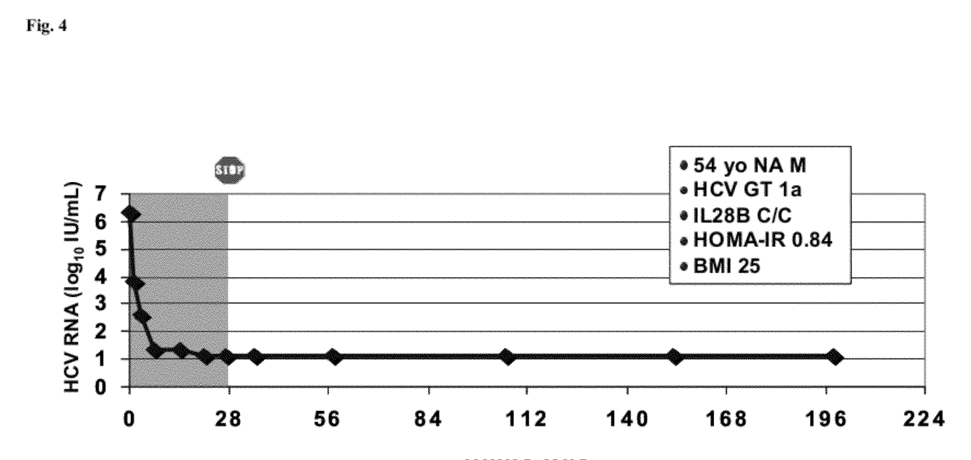

Patient 2, a 54 year old Native American male with GT-1a HCV, had high-level viremia (6.87 log₁₀ IU/mL), Stage 1 fibrosis, Grade 2 ALT elevation (237 IU), and C/C IL28B polymorphism. HOMA-IR was <3 and BMI 25 kg/m2. Patient 2 was randomized to PSI-7977 400 mg QD for 28 days and also elected to discontinue PEG-IFN and RBV at day 28 due to interferon intolerance issues. He experienced rapid antiviral response with HCV RNA<LOD at day 21. ALT was within normal limits (WNL) by day 7 (as shown in FIG. 4). Interferon was reduced to 90 μg/week at day 14; at day 28 he discontinued all therapy due to worsening depression. HCV RNA remained <LOD 24 weeks following 4 weeks of PSI-7977/SOC (SVR24).

Patient 3 with C/C IL28B polymorphism and GT-1 HCV received PSI-7977 400 mg QD for 28 days; elected to discontinue SOC at week 12. A 42 yo Caucasian male with HCV RNA 7.27 log₁₀ IU/L, BMI 27, and HOMA-IR 4.02, he experienced a rapid reduction in HCV RNA<LOD at day 14. ALT activity was WNL throughout treatment. HCV RNA remains <LOD 12 weeks after the last dose of interferon (SVR12). The patient continued in follow up.

Stratification by IL28B status resulted in cohorts balanced for baseline HCV RNA and other traditional predictors of response. IL28B genotype distribution was found to vary geographically within the U.S. in the study. Significant and consistent antiviral activity was observed following 28 days of SOC plus PSI-7977, a nucleotide analog in development for HCV. At such effective doses, and with no on-treatment breakthrough, the potential influence of genetically-controlled interferon susceptibility may not play a role in early antiviral measures of effectiveness.

These results demonstrate that SVR in HCV GT-1 is achievable with very short durations of combination therapies using potent direct acting antiviral agents, especially in patients with favorable predictors of response to interferon. Although anecdotal reports of very rapid responders to abbreviated therapy have been discussed, we believe SVR in 3 of the 15 patients who received PSI-7977 400 mg and very brief (4-12 weeks) SOC is notable. Further studies are underway to explore these observations and to evaluate optimal duration of therapy in individuals based on IFN-susceptibility characteristics.

Although a full and complete description is believed to be contained herein, certain patent and non-patent references may include certain essential subject matter. To the extent that these patent and non-patent references describe essential subject matter, these references are hereby incorporated by reference in their entirety. It is understood that the meanings of the incorporated subject matter are subservient to the meanings of the subject matter disclosed herein.

The subject matter of U.S. 61/408,304, filed Oct. 29, 2010 is hereby incorporated by reference.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

1. A method for administering an abbreviated hepatitis C virus (HCV) treatment regimen for an HCV-infected, rs12979860 single nucleotide polymorphism (SNP) (C/C) patient, which comprises:
administering to said patient an effective amount of each of a direct-acting antiviral, pegylated interferon alfa-2a, and ribavirin;
wherein the abbreviated HCV treatment regimen is less than 48 weeks.

2. The method of claim 1, wherein the administering occurs from about 2 weeks to about 12 weeks.

3. The method of claim 1, wherein the administering occurs from about 2 weeks to about 6 weeks.

4. The method of claim 1, wherein the administering occurs from about 2 weeks to about 4 weeks.

5. The method of claim 1, wherein the administering occurs for about 4 weeks.

6. The method of claim 1, wherein the administering occurs for about 2 weeks.

7. The method of claim 1, wherein the patient is infected with HCV genotype (GT) 1, HCV GT 2, HCV GT 4, or HCV GT 5.

8. The method of claim 1, wherein the patient is infected with HCV GT 1.

9. The method of claim 1, wherein the direct-acting antiviral is PSI-7977.

10. The method of claim 9, wherein the rs12979860 SNP C/C patient is administered 100 mg once per day of PSI-7977, 180 μg once per week of peginterferon alfa-2a, and 1000 mg once per day of ribavirin.

11. The method of claim 9, wherein the rs12979860 SNP C/C patient is administered 100 mg once per day of PSI-7977, 180 μg once per week of peginterferon alfa-2a, and 1200 mg once per day of ribavirin.

12. The method of claim 9, wherein the rs12979860 SNP C/C patient is administered 200 mg once per day of PSI-7977, 180 μg once per week of peginterferon alfa-2a, and 1000 mg once per day of ribavirin.

13. The method of claim 9, wherein the rs12979860 SNP C/C patient is administered 200 mg once per day of PSI-7977, 180 μg once per week of peginterferon alfa-2a, and 1200 mg once per day of ribavirin.

14. The method of claim 9, wherein the rs12979860 SNP C/C patient is administered 400 mg once per day of PSI-7977, 180 μg once per week of peginterferon alfa-2a, and 1000 mg once per day of ribavirin.

15. The method of claim 14, wherein the administration lasts for about two weeks to about six weeks.

16. The method of claim 15, wherein upon the end of the administration period the rs12979860 SNP (C/C) patient has an HCV RNA level less than about 15 IU/mL.

17. The method of claim 14, wherein the administration lasts for about 2 weeks to about four weeks.

18. The method of claim 17, wherein upon the end of the administration period the rs12979860 SNP (C/C) patient has an HCV RNA level less than about 15 IU/mL.

19. The method of claim 9, wherein the rs12979860 SNP C/C patient is administered 400 mg once per day of PSI-7977, 90 μg to 180 μg once per week of peginterferon alfa-2a, and 1000 mg or 1200 mg once per day of ribavirin.

20. The method of claim 19, wherein the administration lasts for about two weeks to about six weeks.

21. The method of claim 20, wherein 180 μg of peginterferon alfa-2a is administered in the first two weeks.

22. The method of claim 21, wherein 90 μg of peginterferon alfa-2a is administered during weeks 3 through 6.

23. The method of claim 19, wherein the administration lasts for about two weeks to about four weeks.

24. The method of claim 23, wherein 180 μg of peginterferon alfa-2a is administered in the first two weeks.

25. The method of claim 21, wherein 90 μg of peginterferon alfa-2a is administered during weeks 3 through 4.

26. The method of claim 19, wherein the patient is infected with HCV GT 1, HCV GT 2, HCV GT, 3, HCV GT4, or a combination thereof.

27. The method of claim 26, wherein the patient is infected with HCV GT 1 or HCV GT 2.

28. The method of claim 27, wherein the patient is infected with HCV GT 1.

29. The method of claim 9, wherein the rs12979860 SNP (C/C) patient is administered 400 mg once per day of PSI-7977, 180 μg once per week of peginterferon alfa-2a, and 1000 mg or 1200 mg once per day of ribavirin for two weeks followed by 400 mg once per day of PSI-7977, 90 μg once per week of peginterferon alfa-2a, and 1000 mg or 1200 mg once per day of ribavirin for two weeks.

30. A method of detecting an rs12979860 single nucleotide polymorphism (SNP) in a hepatitis C virus (HCV) infected patient comprising:
detecting the rs12979860 SNP of chromosome 19 in a biological sample from a patient,
wherein an rs12979860 SNP C/C patient is administered an abbreviated treatment regimen with a direct-acting antiviral agent in combination with peginterferon alfa-2a and ribavirin; and
wherein the abbreviated HCV treatment regimen is less than 48 weeks.

31. The method of claim 30, wherein the rs12979860 SNP C/C patient is administered an abbreviated treatment regimen from about 2 weeks to about 12 weeks.

32. The method of claim 1, wherein the rs12979860 SNP C/C patient is administered an abbreviated treatment regimen from about 2 weeks to about 6 weeks.

33. The method of claim 30, wherein the rs12979860 SNP C/C patient is administered an abbreviated treatment regimen from about 2 weeks to about 4 weeks.

34. The method of claim 30, wherein the rs12979860 SNP C/C patient is administered an abbreviated treatment regimen for about 4 weeks.

35. The method of claim 30, wherein the rs12979860 SNP C/C patient is administered an abbreviated treatment regimen for about 2 weeks.

36. The method of claim 30, wherein the patient is infected with HCV genotype (GT) 1, HCV GT 2, HCV GT 4, or HCV GT 5.

37. The method of claim 30, wherein the patient is infected with HCV GT 1.

38. The method of claim 30, wherein the direct-acting antiviral is PSI-7977.

39. The method of claim 38, wherein the rs12979860 SNP C/C patient is administered 100 mg once per day of PSI-7977, 180 μg once per week of peginterferon alfa-2a, and 1000 mg once per day of ribavirin.

40. The method of claim 38, wherein the rs12979860 SNP C/C patient is administered 100 mg once per day of PSI-7977, 180 μg once per week of peginterferon alfa-2a, and 1200 mg once per day of ribavirin.

41. The method of claim 38, wherein the rs12979860 SNP C/C patient is administered 200 mg once per day of PSI-7977, 180 μg once per week of peginterferon alfa-2a, and 1000 mg once per day of ribavirin.

42. The method of claim 38, wherein the rs12979860 SNP C/C patient is administered 200 mg once per day of PSI-7977, 180 μg once per week of peginterferon alfa-2a, and 1200 mg once per day of ribavirin.

43. The method of claim 38, wherein the rs12979860 SNP C/C patient is administered 400 mg once per day of PSI-7977, 180 μg once per week of peginterferon alfa-2a, and 1000 mg once per day of ribavirin.

44. The method of claim 43, wherein the administration lasts for about two weeks to about six weeks.

45. The method of claim 44, wherein upon the end of the administration period the rs12979860 SNP (C/C) patient has an HCV RNA level less than about 15 IU/mL.

46. The method of claim 43, wherein the administration lasts for about 2 weeks to about four weeks.

47. The method of claim 46, wherein upon the end of the administration period the rs12979860 SNP (C/C) patient has an HCV RNA level less than about 15 IU/mL.

48. The method of claim 38, wherein the rs12979860 SNP C/C patient is administered 400 mg once per day of PSI-7977, 90 μg to 180 μg once per week of peginterferon alfa-2a, and 1000 mg or 1200 mg once per day of ribavirin.

49. The method of claim 48, wherein the administration lasts for about two weeks to about six weeks.

50. The method of claim 49, wherein 180 μg of peginterferon alfa-2a is administered in the first two weeks.

51. The method of claim 50, wherein 90 μg of peginterferon alfa-2a is administered during weeks 3 through 6.

52. The method of claim 48, wherein the administration lasts for about two weeks to about four weeks.

53. The method of claim 52, wherein 180 μg of peginterferon alfa-2a is administered in the first two weeks.

54. The method of claim 50, wherein 90 μg of peginterferon alfa-2a is administered during weeks 3 through 4.

55. The method of claim 38, wherein the rs12979860 SNP (C/C) patient is administered 400 mg once per day of PSI-7977, 180 μg once per week of peginterferon alfa-2a, and 1000 mg or 1200 mg once per day of ribavirin for two weeks followed by 400 mg once per day of PSI-7977, 90 μg once per week of peginterferon alfa-2a, and 1000 mg or 1200 mg once per day of ribavirin for two weeks.
